(19) United States
(12) Plant Patent Application Publication
(10) Pub. No.: US 2023/0210036 P1
(43) Pub. Date: Jun. 29, 2023

(54) Hop Plant Named 'Furano 0414B Go'

(50) ;

(71) Applicant: Sapporo Breweries Limited, Tokyo (JP)

(72) Inventors: Seigi Kuji, Tokyo (JP); Yutaka Itoga, Tokyo (JP); Koichiro Koie, Tokyo (JP); Mitsuhiro Uemoto, Tokyo (JP); Yuto Furukawa, Tokyo (JP); Masanobu Goto, Tokyo (JP); Katsuyoshi Shimaoka, Tokyo (JP)

(73) Assignee: Sapporo Breweries Limited, Tokyo (JP)

(21) Appl. No.: 18/086,014

(22) Filed: Dec. 21, 2022

(30) Foreign Application Priority Data
Dec. 24, 2021 (JP) 35915

(51) Int. Cl. A01H 6/00

(52) U.S. Cl. PLT/236

(57) ABSTRACT

A new hop plant particularly distinguished by having a cylindrical to club plant shape, an alpha acids in cones of 8.7%, cohumulone ratio to alpha acids of 20.7%, myrcene ratio to total oil of 63.1% and a farnesene ratio to total oil of 2.9% as well as a fruit and flower flavor, is disclosed.

### CROSS-REFERENCE TO RELATED APPLICATION

This patent application claims the benefit of priority from Japan Plant Breeders Rights Application No. 35915 as filed on Dec. 24, 2021, the contents of which is incorporated herein by reference for all it teaches and discloses.

Genus And species: Humulus lupulus L.

Variety denomination: ‘Furano 0414B Go’.

### BACKGROUND OF THE NEW PLANT

The present invention comprises a new and distinct variety of Hop, botanically known as Humulus lupulus L. and hereinafter referred to by the variety name ‘Furano 0414B Go’. ‘Furano 0414B Go’ is a selection from a controlled cross of the commercial hop female parent ‘Little Star’ (unpatented) and the proprietary male parent ‘980573’ (unpatented).

The commercial female hop line ‘Little Star’ and the proprietary male hop line ‘980573’ were cross-pollinated in 2001 in Kamifurano-cho, Sorachi-gun, Hokkaido, Japan and seeds were obtained. The seeds were sown and plants were grown for evaluation. A plant line was selected in March of 2003 in Kamifurano-cho, Sorachi-gun, Hokkaido, Japan and named ‘Furano 0414B Go’. In 2005, ‘Furano 0414B Go’ was first vegetatively propagated by in Kamifurano, Hokkaido, Japan via vegetative cuttings. ‘Furano 0414B Go’ was found to reproduce true to type in successive generations of asexual propagation via vegetative cuttings in Kamifurano, Hokkaido, Japan.

### SUMMARY

The following are the most outstanding and distinguishing characteristics of this new variety when grown under normal horticultural practices in Kamifurano, Hokkaido, Japan.
1. Cylindrical to club plant shape;
2. An alpha acids in cones of 8.7%;
3. A cohumulone ratio to alpha acids of 20.7%;
4. A myrcene ratio to total oil of 63.1%;
5. A farnesene ratio to total oil of 2.9%; and
6. A fruit and flower flavor.

### DESCRIPTION OF THE PHOTOGRAPHS

This hop plant is illustrated by the accompanying photographs which show the plant's overall plant shape, leaf shape and cone shape.

FIGS. 1 and 3 are of a three-year-old plant grown in Kamifurano, Hokkaido, Japan in August 2020 while FIG. 2 is also of a four-year-old plant grown in Kamifurano, Hokkaido, Japan taken in August 2021. The colors shown are as true as can be reasonably obtained by conventional photographic procedures.

FIG. 1 shows the plant shape of ‘Furano 0414B Go’.

FIG. 2 shows the leaf shape of ‘Furano 0414B Go’ together with that of ‘Little Star’ and ‘980573’ in the left and in the middle, respectively.

FIG. 3 shows the cone shape of ‘Furano 0414B Go’ together with that of ‘Little Star’ and ‘980537’ in the left and in the middle, respectively.

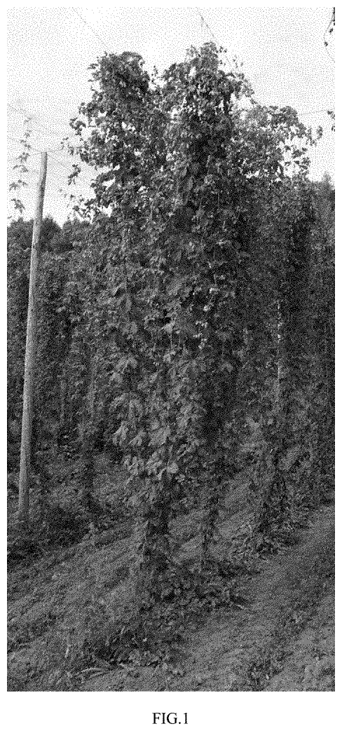

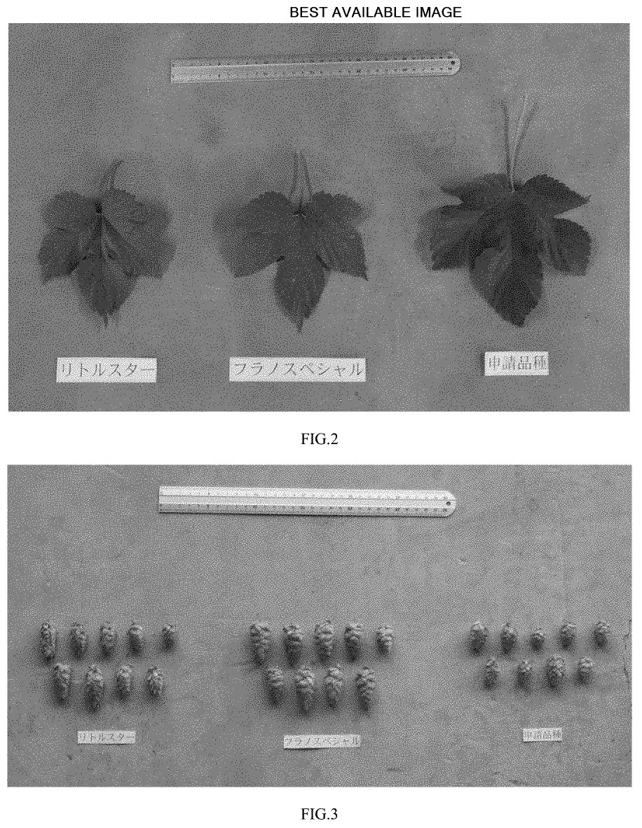

### DESCRIPTION OF THE NEW VARIETY

The following detailed descriptions set forth the distinctive characteristics of ‘Furano 0414B Go’. The data, which define these characteristics, were collected from asexual reproductions carried out in Kamifurano, Hokkaido, Japan. Data was collected on three-year-old plants in Kamifurano, Hokkaido, Japan in 2020. Color references are to Japan Horticultural Plant Color Chart (1987, Japan Color Research Institute).
Classification:
   Family.—Cannabaceae.
   Classification.—Humulus lupulus L.
   Common name.—Hop.
Parents:
   Female parent.—‘Little Star’ (unpatented).
   Male parent.—‘980573’ (unpatented).
Propagation:
   Type.—Vegetative cuttings or rhizomes.
Plant description:
   Plant and growth habit.—The plant growth type is normal, not dwarf.
   Plant shape.—Cylindrical to club.
   Plant height.—Hop plants were grown at a trellis of 5.5 m high.
   Anthocyanin.—Anthocyanin coloration of the main bine is medium.
   Anthocyanin color.—Dark red purple (9510).
Lateral branch description:
   Length.—The average length of lateral branches is 97.9 cm, ranged between 42.1-166.0 cm.
   Diameter.—The average diameter of lateral branches is 0.35 cm, ranged between 0.30-0.38 cm.
   Internode length.—The average internode length of lateral branches is 11.6 cm, ranged between 6.0-25.0 cm.
   Color, fully developed (mature).—Upper surface is bright yellow green (3504), and lower surface is strong red purple (9214).
Foliage description:
   Arrangement.—Opposite.
   Length.—The average length of foliage is 15.3 cm, ranged between 13.0-17.0 cm.
   Width.—The average width of foliage is 15.2 cm, ranged between 12.5-16.5 cm.
   Shape.—Palmate, 3 to 5 lobes.
   Apex.—Cuspidate.
   Base.—Cordate.
   Margin.—Serrate.
   Texture.—Upper surface: Blistering of upper side of blade is slightly strong.
   Color.—Fully expanded (mature) leaves: Upper surface: Dark green (3707).
Petiole:
   Length.—The average length of petioles is 6.9 cm, ranged between 6.0-8.0 cm.
   Diameter.—The average diameter of petioles is 0.34 cm, ranged between 0.31-0.38 cm.
   Color.—Upper and lower surfaces: Light yellow green (3503).
Flower description:
   Cones.—Number of cones per lateral branch from middle of plant is few (average: 34.9 cones/branch, ranged from 20-49). Number of cones per node of lateral branches from middle of plant is slightly few (average: 6.4 cones/node, ranged from 3.5-12.3). Number of cones per lateral branch from upper third of plant is quite many (average: 110.2 cones/branch ranged between 26-420).
   Time of flowering.—Slightly early (early July).
   Time of picking maturity of cones.—Medium (end of August).
Inflorescence buds:
   Length.—The average length of cones is 3.3 cm, ranged between 2.4-3.8 cm.
   Diameter.—The average diameter of cones is 2.0 cm, ranged between 1.5-2.4 cm.
   Shape.—The shape of cones is medium ovate, degree of opening bracts is slightly opened.
Spike bracts:
   Length.—The average length of spike bracts is 1.3 cm, ranged between 1.0-1.7 cm.
   Width.—The average width of spike bracts is 1.1 cm, ranged between 0.9-1.2 cm.
   Shape.—The shape is obtuse, and the width/length ratio is very large (average width/length ratio of bracts is 0.86, ranged between 0.71-1.09).
   Apex.—The shape of apex is apiculate, and the length of apex of bract is medium.
   Margin.—Entire.
   Color.—Deep yellow green (3307).
Bracteoles:
   Length.—The average length of bracteoles is 1.1 cm, ranged between 1.0-1.3 cm.
   Width.—The average width of bracteoles is 0.7 cm, ranged between 0.6-0.8 cm.
   Shape.—The average width/length ratio is 0.59, ranged between 0.50-0.73.
Pedicels: Not observed:
Reproductive organs:
   Stamens.—Not observed.
   Pistils.—Not observed.
Disease and pest/insect resistance: Not resistant to either downy mildew or powdery mildew.
Brewing characteristics:
   Alpha acids content in cones.—8.7%.
   Ratio of beta acids to alpha acids.—1.00.
   Ratio of co-alpha to all alpha acids.—0.21.
   Humulene ratio to total oil.—0.20.
   Caryophyllene ratio to total oil.—0.10.
   Humulene ratioto caryophyllene.—2.02.
   Myrcene ratioto total oil.—0.63.
   Farnesene ratioto total oil.—0.03.
   Linalool ratioto total oil.—0.01.
   Remaining rate of alpha acids after 6 months storage at room temperature.—56.2%.
   Yield per hectare.—1.86 t/ha.
   Flavor profile.—Fruits, flower.

### COMPARISON WITH PARENTAL LINES AND KNOWN VARIETY

‘Furano 0414B Go’ is a distinct variety of hop. ‘Furano 0414B Go’ is distinguished from its female parent ‘Little Star’ and the male parent ‘980573’ as described in Table 1:

TABLE 1
Comparison with Parental Lines

| Characteristic | Variety | Female parent | Male Parent |
|---|---|---|---|
| | ‘Furano 0414B | ‘Little Star’ | ‘980573’ |
| | Go’ | | |
| Flower | Produce female | Produce female | Does not produce |
| | flowers and | flowers and cones | any cones, but |
| | cones | | produce male |
| | | | flower |
| Alpha acids in | 8.7% | 4.9% | — |
| cones | | | |
| Cohumulone | 20.7% | 25.3% | — |
| ratioto alpha | | | |
| acids | | | |
| Farnesene ratioto | 2.9% | 17.9% | — |
| total oil | | | |

‘Furano 0414B Go’ is distinguished from the commercial hop variety ‘Furano K906901060 go’, commercially named ‘Furano Magical’ (U.S. Publication No. US20180098474). Differences between the two varieties are described in Table 2:

TABLE 2
Comparison with Similar Variety

| | Variety | Commercial line |
|---|---|---|
| Characteristic | ‘Furano 0414B Go’ | ‘Furano K906901060 go’ |
| Alpha acids in cones | 8.7% | 5.4% |
| Cohumulone ratio to | 20.7% | 16.6% |
| alpha acids | | |
| Myrcene ratioto total oil | 63.1% | 31.7% |
| Farnesene ratioto total | 2.9% | 0.8% |
| oil | | |

We claim:

1. A new and distinct variety of hop plant named ‘Furano 0414B Go’ as illustrated and described herein.
